(19) United States
(12) Patent Application Publication
(10) Pub. No.: US 2022/0395337 A1
(43) Pub. Date: Dec. 15, 2022

(54) SURGERY ASSISTANCE SYSTEM, OPERATING METHOD FOR SURGERY ASSISTANCE SYSTEM, AND CONTROL DEVICE OF SURGERY ASSISTANCE SYSTEM

(71) Applicant: OLYMPUS CORPORATION, Tokyo (JP)

(72) Inventor: Katsuhiko YOSHIMURA, Koganei-shi (JP)

(73) Assignee: OLYMPUS CORPORATION, Tokyo (JP)

(21) Appl. No.: 17/890,635

(22) Filed: Aug. 18, 2022

Related U.S. Application Data
(63) Continuation of application No. PCT/JP2020/010184, filed on Mar. 10, 2020.

(51) Int. Cl. A61B 34/00; A61B 1/313; A61B 1/00

(52) U.S. Cl. CPC A61B 34/25; A61B 1/3132; A61B 1/0005; A61B 2034/107

(57) ABSTRACT

A surgery assistance system includes: an endoscope; a display configured to display an image from the endoscope; a treatment tool that includes an end effector at a distal end; an input device that inputs an instruction to the end effector; and a processor connected to the endoscope, the display, the treatment tool, and the input device, wherein the processor is configured to detect a distal end position of the end effector based on the instruction, record the detected distal end position, and estimate a first treatment surface from a plurality of recorded distal end positions.

### CROSS-REFERENCE TO RELATED APPLICATIONS

This application is a continuation application based on a PCT Patent Application No. PCT/JP2020/010184, filed on Mar. 10, 2020, the entire content of which is hereby incorporated by reference.

### BACKGROUND

### Technical Field

The present invention relates to a surgery assistance system that performs treatment through a hole formed in the abdominal wall or the like, an operating method for the surgery assistance system, and a control device of the surgery assistance system.

### Background Art

Conventionally, in laparoscopic surgery, a method of performing treatment by inserting a treatment tool or an endoscope through separate holes (openings) opened in the abdominal wall has been used. A surgery assistance system has been devised to support the surgeon by presenting a virtual image generated from a model (shape data or image) of the target organ created by a preoperative plan using CT images to the surgeon during the operation.

The surgery assistance system described in Japanese Patent Publication No. 4,698,966 (hereinafter referred to as Patent Document 1) supports the surgeon performing the procedure by presenting a virtual image according to the progress of the procedure to the surgeon. The surgery assistance system described in Patent Document 1 provides a virtual image suitable for surgical support such as a resection surface set before surgery and a vessel in the vicinity of the resection surface in real time during the procedure.

In the surgery assistance system described in Patent Document 1, the excised surface displayed as a virtual image is only displayed as the preset one before the operation even if the situation changes during the operation, which does not provide the necessary information to the surgeon.

### SUMMARY

The present invention provides a surgery assistance system that can estimate the excised surface to be actually excised and present it to the surgeon.

According to a first aspect of the present invention, a surgery assistance system includes: an endoscope; a display configured to display an image from the endoscope; a treatment tool that includes an end effector at a distal end; an input device that inputs an instruction to the end effector; and a processor connected to the endoscope, the display, the treatment tool, and the input device, wherein the processor is configured to detect a distal end position of the end effector based on the instruction, record the detected distal end position, and estimate a first treatment surface from a plurality of recorded distal end positions.

According to a second aspect of the present invention, an operating method for a surgery assistance system, which includes a treatment tool equipped with an end effector at a distal end, includes: an anatomical information acquisition step of acquiring anatomical information of a target organ; a treatment point position detection step of detecting a distal end position of the end effector; a treatment point position recording step of recording the distal end position that has been detected; an estimated excision surface estimation step of estimating a first treatment surface from the distal end position that has been recorded; and a related information presentation step of presenting the anatomical information related to the first treatment surface.

According to a third aspect of the present invention, a control device of a surgery assistance system, which includes a treatment tool equipped with an end effector at a distal end, includes a processor that performs: an anatomical information acquisition step of acquiring anatomical information of a target organ; a treatment point position detection step of detecting a distal end position of the end effector; a treatment point position recording step of recording the distal end position that has been detected; an estimated excision surface estimation step of estimating a first treatment surface from the distal end position that has been recorded; and a related information presentation step of presenting the anatomical information related to the first treatment surface.

The surgery assistance system according to the present invention can estimate the excised surface to be actually excised and present it to the surgeon.

### BRIEF DESCRIPTION OF THE DRAWINGS

FIG. 1 is a diagram showing an overall configuration of a surgery assistance system according to a first embodiment of the present invention.

FIG. 2 is a hardware configuration diagram of the surgery assistance system.

FIG. 3 shows a model of the target organ and surrounding organs created in the preoperative plan.

FIG. 4 is a diagram showing an insertion portion of an endoscope inserted into the abdominal cavity and a target organ.

FIG. 5 is a display image generated by a control device from an image captured by an imaging portion of an endoscope.

FIG. 6 is a control flowchart of a control portion of the surgery assistance system.

FIG. 7 is an explanatory diagram of registration performed by the control portion of the surgery assistance system.

FIG. 8 is a diagram in which a virtual image of a vessel visualized as a three-dimensional image is superimposed and displayed on a display image.

FIG. 9 is an example of a displayed image in which anatomical information related to the estimated excision surface is superimposed and displayed.

FIG. 10 is a control flowchart of a control portion of the surgery assistance system according to a second embodiment of the present invention.

FIG. 11 is a control flowchart of a control portion of the surgery assistance system according to a third embodiment of the present invention.

FIG. 12 is a diagram showing the positions of recorded treatment points and treatment means.

FIG. 13 is a virtual image of a vessel after a reregistration step.

### DETAILED DESCRIPTION OF THE PREFERRED EMBODIMENTS

### First Embodiment

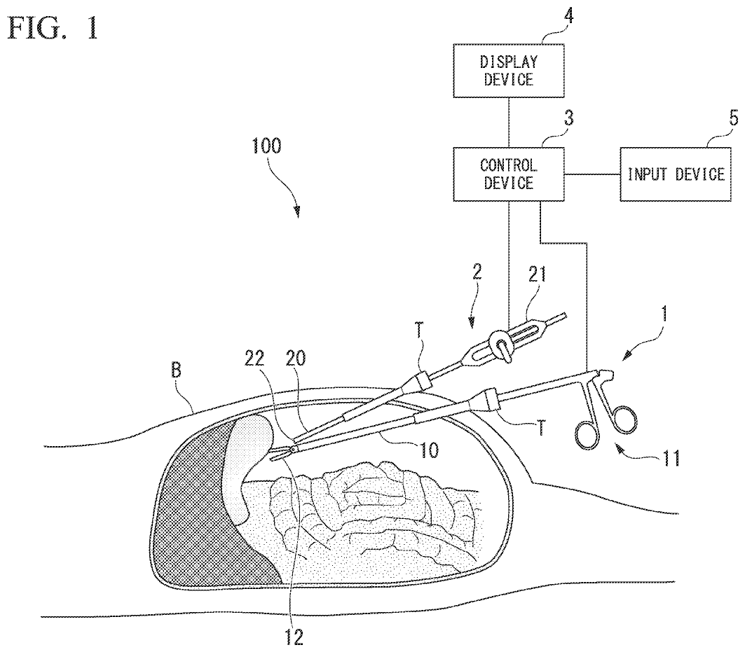

A first embodiment of the present invention will be described with reference to FIGS. 1 to 6.

FIG. 1 is a diagram showing the overall configuration of a surgery assistance system 100 according to the present embodiment.

### [Surgery Assistance System 100]

As shown in FIG. 1, the surgery assistance system 100 includes a treatment tool 1, an endoscope 2, a control device 3, a display device 4, and an input device 5. The surgery assistance system 100 is a system that supports a procedure performed by inserting the treatment tool 1 or the endoscope 2 through separate holes (openings) opened in the abdominal wall in laparoscopic surgery.

The treatment tool 1 has a long insertion portion 10 that can be inserted into the abdominal cavity of the patient, and an operation portion 11 provided on the proximal end side of the insertion portion 10. The surgeon passes the insertion portion 10 through a trocar T punctured in the abdomen B of the patient and introduces the insertion portion 10 into the abdominal cavity. Depending on the type of treatment and the condition of the affected area, the surgeon may introduce a plurality of treatment tools 1 into the abdominal cavity. The treatment tool 1 is an energy device. The treatment tool 1 is connected to the control device 3 and energy is supplied from the control device 3.

The insertion portion 10 has a treatment portion 12 at the distal end thereof for treating the affected portion of the patient. The treatment portion 12 is formed in the shape of forceps. The treatment portion 12 energizes the affected area with the energy supplied from the energy supply source. The treatment portion 12 includes two operation modes, an “incision mode” for incising the affected area and a “hemostatic mode” for stopping the bleeding of the affected area. These two operation modes are realized by appropriately adjusting the magnitude and frequency of the current. Although the forceps-shaped treatment portion 12 is disclosed in the embodiment, the same applies to a monopolar type treatment tool.

The operation portion 11 is a member that operates the treatment portion 12. The operation portion 11 has a handle. The surgeon can open and close the treatment portion 12 by moving the handle relative to other parts of the operation portion 11.

The endoscope 2 has a long and rigid insertion portion 20 that can be inserted into the abdominal cavity of the patient, and an operation portion 21. The surgeon passes the insertion portion 20 through the trocar T punctured in the abdomen B of the patient and introduces the insertion portion 20 into the abdominal cavity.

The insertion portion 20 has an imaging portion 22 at the distal end. The imaging portion 22 has a lens and an imaging element for photographing the inside of the abdomen of the patient. In the insertion portion 20 introduced into the abdominal cavity, the imaging portion 22 is arranged at a position in the abdomen where the affected portion to be treated can be photographed. The imaging portion 22 may have an optical zoom or an electronic zoom function.

The operation portion 21 is a member operated by the surgeon. The surgeon can change the position and orientation of the imaging portion 22 of the endoscope 2 by moving the endoscope 2 with the operation portion 21. The insertion portion 20 may further have a curved portion. By bending the curved portion provided in a part of the insertion portion 20, the position and orientation of the imaging portion 22 can be changed.

Inside the operation portion 21, a control signal line for controlling the imaging portion 22, a transmission signal for transferring the captured image captured by the imaging portion 22, and the like are wired.

As shown in FIG. 1, the control device 3 receives the captured image captured by the imaging portion 22 of the endoscope 2 and transfers it to the display device 4 as a display image.

The control device 3 is a program-executable device (computer) equipped with a processor such as a CPU (Central Processing Unit) and hardware such as a memory. The function of the control device 3 can be realized as a function of the program (software) by reading and executing the program for controlling the processor by the control device 3. In addition, at least a part of the control device 3 may be configured by a dedicated logic circuit or the like. Further, the same function can be realized by connecting at least a part of the hardware constituting the control device 3 with a communication line.

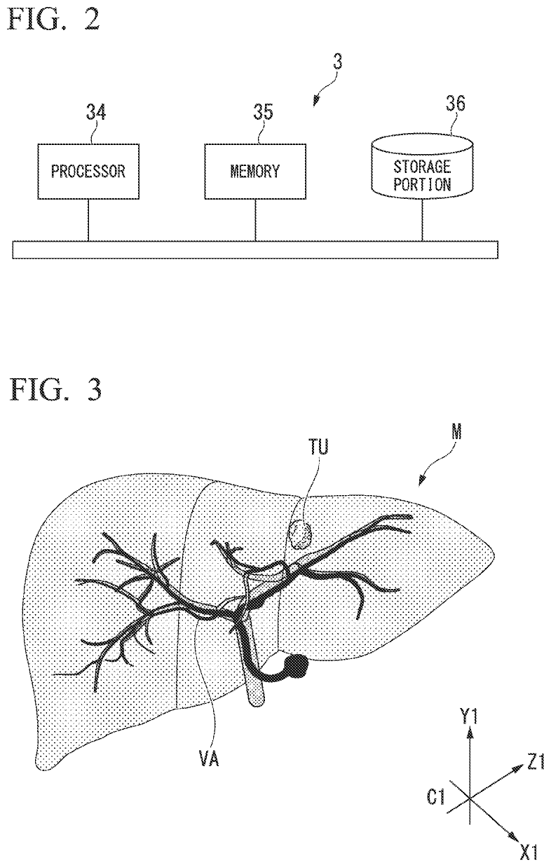

FIG. 2 is a diagram showing an overall configuration example of the control device 3.

The control device 3 has a processor 34, a memory 35 capable of reading a program, and a storage portion 36. The program provided to the control device 3 for controlling the operation of the control device 3 is read into the memory 35 and executed by the processor 34.

The storage portion 36 is a non-volatile recording medium that stores the above-mentioned program and necessary data. The storage portion 36 is composed of, for example, a ROM, a hard disk, or the like. The program recorded in the storage portion 36 is read into the memory 35 and executed by the processor 34.

The control device 3 receives the input data from the endoscope 2 and transfers the input data to the processor 34 or the like. Further, the control device 3 generates data, a control signal, and the like for the endoscope 2 and the display device 4 based on the instruction of the processor 34.

The control device 3 receives the captured image as input data from the endoscope 2 and reads the captured image into the memory 35. Based on the program read into the memory 35, the processor 34 performs image processing on the captured image. The captured image that has undergone image processing is transferred to the display device 4 as a display image.

The control device 3 performs image processing such as image format conversion, contrast adjustment, and resizing processing on the captured image to generate a display image. Further, the control device 3 performs image processing for superimposing a virtual image such as an estimated excision surface, which will be described later, on the display image.

Here, the control device 3 is not limited to the device provided in one hardware. For example, the control device 3 may be configured by separating the processor 34, the memory 35, the storage portion 36, and the input/output control portion 37 as separate hardware, and connecting the hardware to each other via a communication line. Alternatively, the control device 3 may be implemented as a cloud system by separating the storage portion 36 and connecting it with a communication line.

The control device 3 may further have a configuration other than the processor 34, the memory 35, and the storage portion 36 shown in FIG. 2. For example, the control device 3 may further have an image calculation portion that performs a part or all of the image processing and the image recognition processing performed by the processor 34. By further having the image calculation portion, the control device 3 can execute specific image processing and image recognition processing at high speed. Further, it may further have an image transfer portion that transfers the display image from the memory 35 to the display device 4.

The display device 4 is a device that displays the display image transferred by the control device 3. The display device 4 has a know-n monitor 41 such as an LCD display. The display device 4 may have a plurality of monitors 41. The display device 4 may include a head-mounted display or a projector instead of the monitor 41.

The monitor 41 can also display a GUI (Graphical User Interface) image generated by the control device 3 as a GUI. For example, the monitor 41 can display control information and the attention alerts from the surgery assistance system 100 to the surgeon by GUI. Further, when the control device 3 requires information input from the surgeon, the display device 4 can also display a message prompting the input device 5 to input information and a GUI display necessary for information input.

The input device 5 is a device in which the surgeon inputs an instruction or the like to the control device 3. The input device 5 is composed of each or a combination of known devices such as a touch panel, a keyboard, a mouse, a stylus, a foot switch, and a button. The input of the input device 5 is transmitted to the control device 3. For example, the above-mentioned “incision mode” and “hemostatic mode” are also input via the input device 5.

### [Operation of Surgery Assistance System 100]

Next, the operation and operating method of the surgery assistance system 100 will be described with reference to FIGS. 3 to 9 by taking laparoscopic surgery with the liver L as a target organ as an example.

Medical staff (including the surgeon) prepares anatomical information of the target organ (liver L) before laparoscopic surgery. Specifically, the medical staff creates three-dimensional shape data (model coordinate system (first coordinate system) C1) of the target organ (liver L) and the organs located around the target organ (peripheral organ) as anatomical information by using a known method from the image information of the diagnosis result such as CT. MRI, and ultrasound of the patient in advance.

FIG. 3 is a model M of the target organ (liver L) and surrounding organs (gallbladder G) created as the above-mentioned anatomical information. The model M is constructed on three-dimensional coordinates (X1 axis, Y1 axis, Z1 axis) in the model coordinate system C1. The anatomical information includes vessel information of the target organ (liver L), position coordinates of the tumor TU, and the like. The vessel information is the type of vessel, the position coordinates of the vessels (three-dimensional coordinates in the model coordinate system C1), and the like. Further, the model M includes the position coordinates (three-dimensional coordinates in the model coordinate system C1) of the tumor TU to be removed by laparoscopic surgery. As shown in FIG. 3, the model M can be displayed on the display device 4 as a three-dimensional image.

The created model M of the target organ is recorded in the storage portion 36 of the control device 3 (anatomical information acquisition step). The model M may be created by an external device other than the surgery assistance system 100, and the surgery assistance system 100 may acquire the created model M from the external device.

The control device 3 extracts and stores a plurality of feature points F in the model M (feature point extraction step). The plurality of feature points F are extracted using a known method for extracting feature points. The plurality of feature points F are specified together with the three-dimensional coordinates in the model coordinate system C1 together with the feature amount calculated according to a predetermined reference suitable for expressing the feature, and are stored in the storage portion 36. The extraction and recording of the plurality of feature points F may be performed preoperatively or intraoperatively.

Next, the operation of the surgery assistance system 100 during laparoscopic surgery will be described. The surgeon provides a plurality of holes (openings) for installing the trocar T in the abdomen of the patient, and punctures the trocar T in the holes. Next, the surgeon passes the insertion portion 10 of the treatment tool 1 through the trocar T punctured in the abdomen of the patient, and introduces the insertion portion 10 into the abdominal cavity.

Next, a scopist operates the endoscope 2 to pass the insertion portion 20 of the endoscope 2 through the trocar T punctured in the abdomen of the patient, and introduces the insertion portion 20 into the abdominal cavity.

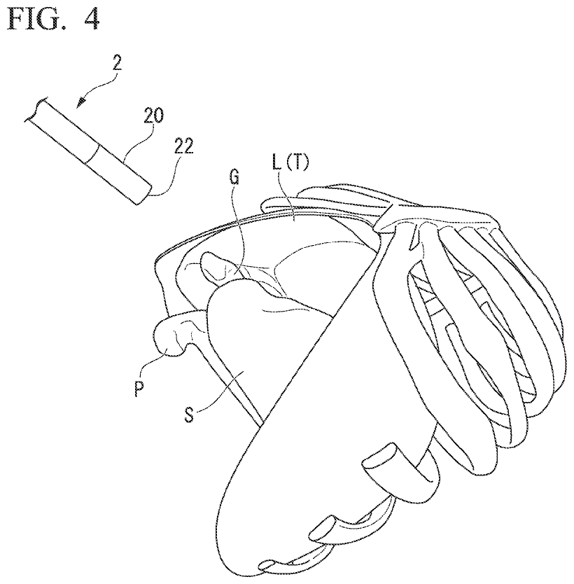

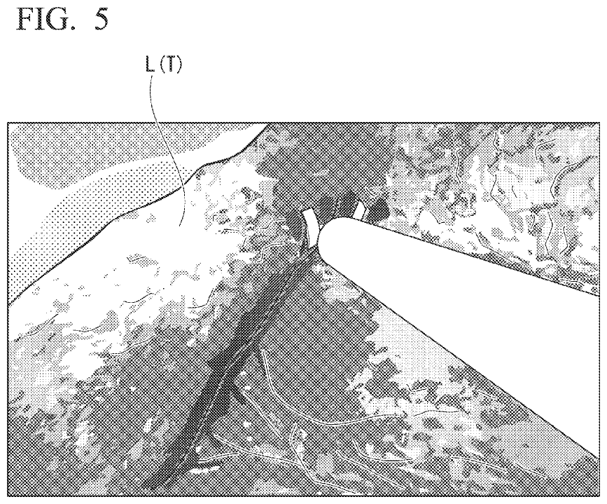

FIG. 4 is a diagram showing the insertion portion 20 of the endoscope 2 inserted into the abdominal cavity and the target organ T. FIG. 5 is a display image generated by the control device 3 from the captured image captured by the imaging portion 22. The three-dimensional coordinate system of the display space displayed by the display image is referred to as the display coordinate system (second coordinate system) C2. Generally, it coincides with the world coordinate system with a certain part on the base end side of the endoscope as the origin (reference) in the space of the actual operating room.

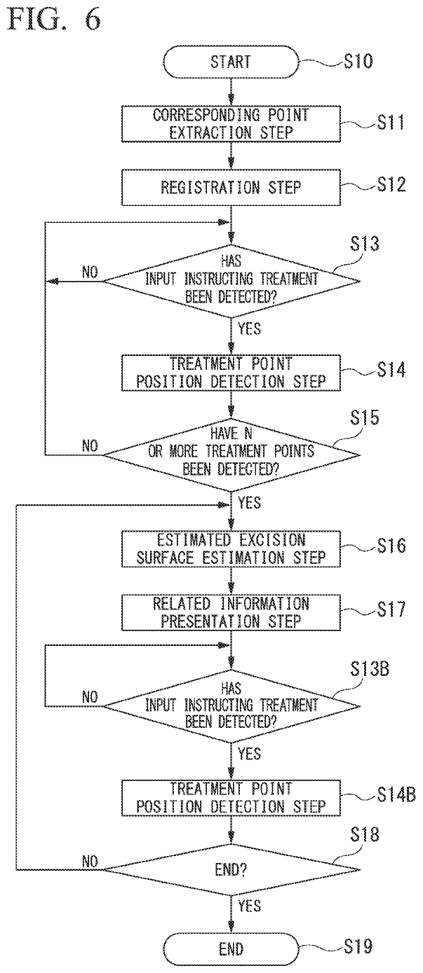

Hereinafter, a description will be given according to the control flowchart of the control device 3 shown in FIG. 6. As shown in FIG. 6, when the control device 3 is activated, the control device 3 starts control after performing initialization (step S10). Next, the control device 3 executes step S11.

In step S11, the control device 3 extracts a plurality of corresponding points A corresponding to the plurality of feature points F in the display image (corresponding point extraction step). The control device 3 extracts the corresponding point A in the display image based on the feature amount of the feature point F stored in the storage portion 36 in advance. For the extraction step, a method appropriately selected from known template matching methods and the like is used. The three-dimensional coordinates in the display coordinate system C2 of the extracted corresponding point A are stored in the storage portion 36.

The surgeon may directly specify the corresponding point A corresponding to the feature point F. For example, the surgeon may move the treatment portion 12 at the distal end of the treatment tool 1 to the corresponding point A corresponding to the feature point F, and the control device 3 may recognize the position of the treatment portion 12 (position in the display coordinate system C2) and extract the corresponding point A. Next, the control device 3 executes step S12.

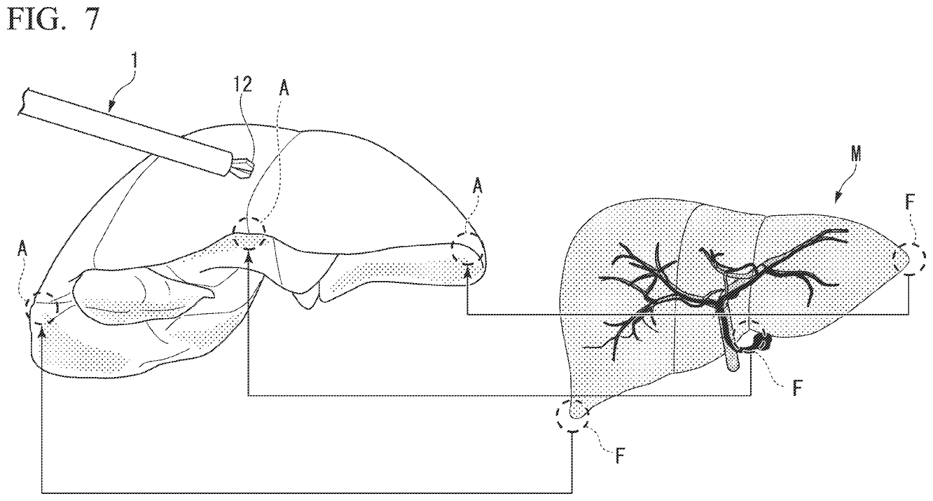

FIG. 7 is an explanatory diagram of registration.

In step S12, the control device 3 makes a correspondence (performs registration) between the model coordinate system C1 of the model M and the display coordinate system C2 of the display space displayed by the display image, based on a plurality of feature points F and a plurality of correspondence points A (registration step). For registration, a method appropriately selected from known coordinate conversion methods and the like is used. For example, the control device 3 performs registration by calculating a correspondence that converts a coordinate position in the model coordinate system C1 into a coordinate position in the display coordinate system C2.

When the registration step is completed, the control device 3 can convert the coordinate position of the model M in the model coordinate system C1 to the coordinate position in the display coordinate system C2 of the display space. Next, the control device 3 executes step S13.

In step S13, the control device 3 detects an input instructing a treatment. In the present embodiment, the control device 3 detects an input instructing energization of the “incision mode” or the “hemostatic mode” from the input device 5. The control device 3 waits until it detects an input that instructs treatment. When the control device 3 detects the input instructing the treatment, the control device 3 executes step S14.

In step S14, the control device 3 detects the position of the treatment point P treated by the treatment tool 1 based on the treatment instruction (treatment point position detection step). In the present embodiment, since the treatment tool 1 is an energy device that energizes from the treatment portion 12 at the distal end, the treatment point P is a portion treated by the treatment portion 12 at the distal end of the treatment tool 1.

The control device 3 detects the three-dimensional coordinates of the treatment point P in the display coordinate system C2. For the detection of the position of the treatment point P, a method appropriately selected from known position detection methods and the like is used. For example, a sensor for detecting the insertion angle and the insertion amount is attached to the trocar T, and the position of the treatment point P may be detected based on the position of the distal end of the endoscope 2 or the treatment portion 12 of the treatment tool 1 detected by the sensor. Further, a position sensor is attached near the treatment portion 12 of the treatment tool 1 and the distal end of the endoscope 2, and the position of the treatment point P may be detected based on the relative position between the treatment portion 12 and the distal end of the endoscope 2 detected by the sensor. Further, the control device 3 may detect the position of the treatment point P by detecting the position of the treatment portion 12 on the display screen by image processing. In either case, the position of the detected treatment point P is converted into three-dimensional coordinates in the display coordinate system C2. The detected position of the treatment point P is recorded in the storage portion 36 (treatment point position recording step). Next, the control device 3 executes step S15.

In step S15, the control device 3 confirms that the treatment point P has been detected by a predetermined value N or more. When the number of detected treatment points P is not equal to or greater than the predetermined value N, the control device 3 executes step S13 again. When the treatment point P is detected by a predetermined value N or more, the control device 3 executes step S16. The predetermined value N needs to be at least 3 or more in order to estimate the estimated excision surface S. The larger the predetermined value N, the better the accuracy of estimating the estimated excision surface S.

In step S16, the control device 3 estimates the estimated excision surface S from the positions of the plurality of recorded treatment points P (estimated excision surface estimation step). The estimated excision surface (first treatment surface) S is a surface including a treatment point where energization treatment is estimated to be performed thereafter, and is estimated based on the positions of a plurality of treatment points P. For the estimation of the estimated excision surface S, a method appropriately selected from known surface estimation methods and the like is used. For example, the control device 3 may calculate the least squares curved surface including the positions of the plurality of recorded treatment points P, and use the least squares curved surface as the estimated excision surface S. The estimated excision surface S is stored in the storage portion 36. Next, the control device 3 executes step S17.

In step S17, the control device 3 displays the anatomical information related to the estimated excision surface S on the display image (related information presentation step). The anatomical information related to the estimated excision surface S is the anatomical information included in the model M acquired before the operation, and is, for example, position information of the tumor TU near the estimated excision surface S and the vessel information of the vessel near the estimated excision surface S.

The anatomical information related to the estimated excision surface S may be displayed as text information on the GUI display of the displayed image. The control device 3 can display the type of the vessel in the vicinity of the estimated excision surface S as a text, and can transmit the vessel in the vicinity of the estimated excision surface S to the surgeon.

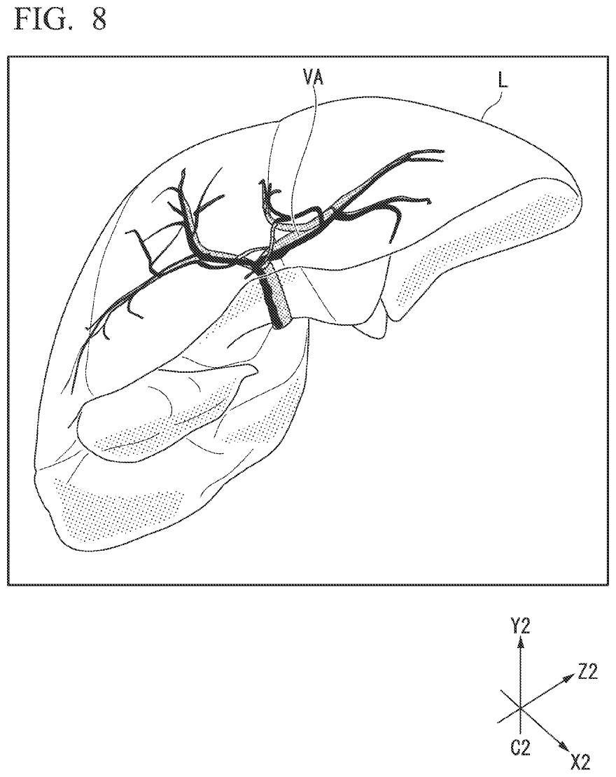

The anatomical information related to the estimated excision surface S may be superimposed and displayed on the display image as a virtual image visualized as a three-dimensional image. FIG. 8 is a diagram in which a virtual image VA of a vessel visualized as a three-dimensional image is superimposed and displayed on a display image. The virtual image VA of the vessel is created based on the position coordinates in the display coordinate system C2 of the vessel converted from the position coordinates in the model coordinate system C1 of the vessel included in the model M. Therefore, the position and size of the virtual image VA of the vessel is the position and size relative to the target organ T (liver L) displayed in the displayed image.

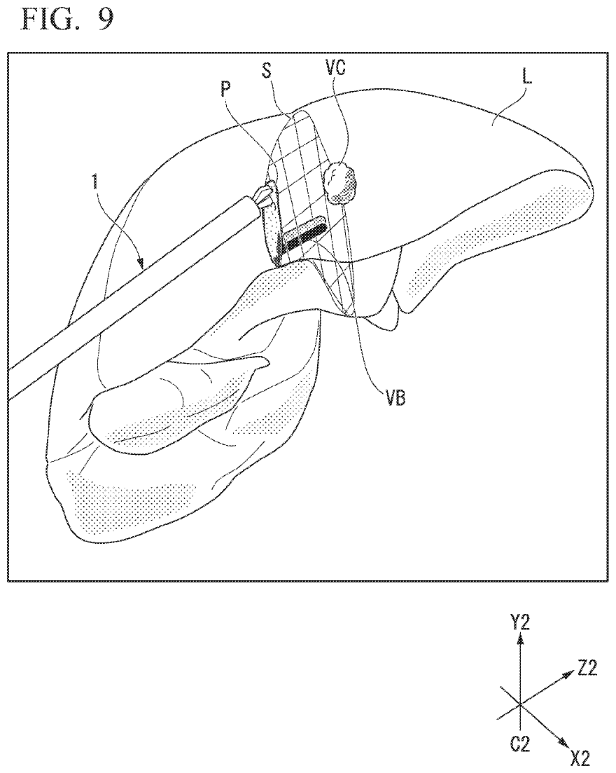

FIG. 9 is an example of a display image in which anatomical information related to the estimated excision surface S is superimposed and displayed.

In the display image shown in FIG. 9, the estimated excision surface S, the virtual image VB of the vessel crossing the estimated excision surface S, and the virtual image VC of the tumor TU are superimposed and displayed on the target organ T (liver L). Instead of the virtual image VA of the entire vessel, the virtual image VB of the vessel that is a part of the vessel and crosses the estimated excision surface S is superimposed and displayed on the display image.

As shown in FIG. 9, the surgeon confirms the actual target organ T (liver L) displayed on the display screen, the estimated excision surface S, and the virtual image VC of the tumor TU together with the estimated excision surface S, so that the positional relationship with the tumor TU can be quickly grasped. If necessary, the surgeon changes the position of the treatment point for subsequent excision.

As shown in FIG. 9, the surgeon can quickly grasp the vessel at the position of the treatment point to be excised thereafter by confirming the estimated excision surface S and the virtual image VB of the vessel together. Since the virtual image VB of the vessel crossing the estimated excision surface S is superimposed and displayed on the display image instead of the virtual image VA of the entire vessel, the surgeon can easily confirm only the vessel information related to the estimated excision surface S. If necessary, the surgeon changes the position of the treatment point for subsequent excision.

The control device 3 then executes step S13B and step S14B. Step S13B and step S14B are the same processes as in step S13 and step S14, and detect and record a new treatment point P. The control device 3 then executes step S18. In step S18, it is determined whether the control device 3 ends the control. When the control is not terminated, the control device 3 executes step S16 again. In step S16, the control device 3 estimates the estimated excision surface S by adding the treatment points P newly detected in steps S13B and S14B. When terminating the control, the control device 3 then performs step S19 to end the control.

According to the surgery assistance system 100 according to the present embodiment, the estimated excision surface S can be estimated from the treatment status of the surgeon, and the position information of the tumor TU related to the estimated excision surface S and vessel information related to the estimated excision surface S can be quickly grasped. In the past, acquiring such information depended on the knowledge and experience of the surgeon. According to the surgery assistance system 100 of the present embodiment, the surgeon can grasp these more accurately and quickly. As a result, the procedure becomes more efficient and the procedure time is reduced.

Although the first embodiment of the present invention has been described in detail with reference to the drawings, the specific configuration is not limited to this embodiment and includes design changes and the like within a range not deviating from the gist of the present invention. In addition, the components shown in the above-described embodiment and the modifications shown below can be appropriately combined and configured.

### Modification 1

For example, in the above embodiment, the anatomical information related to the estimated excision surface S is presented to the surgeon by displaying it on the displayed image, but the presentation mode of the related information is not limited to this. The anatomical information related to the putative excision surface S may be presented to the surgeon, for example, by voice.

### Second Embodiment

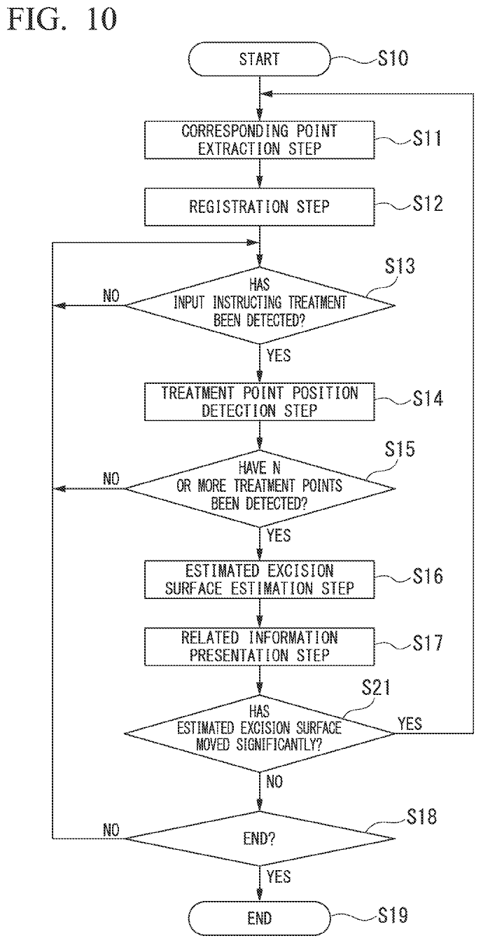

The second embodiment of the present invention will be described with reference to FIG. 10. In the following description, the same reference numerals will be given to the configurations common to those already described, and duplicate description will be omitted.

Similar to the surgery assistance system 100 according to the first embodiment, a surgery assistance system 100B according to the present embodiment includes a treatment tool 1, an endoscope 2, a control device 3, a display device 4, an input device 5, and the like. The surgery assistance system 100B differs from the surgery assistance system 100 according to the first embodiment only in the control performed by the control device 3. Hereinafter, a description will be given according to the control flowchart of the control device 3 shown in FIG. 10. The control from step S10 to step S17 is the same as that of the first embodiment.

In the present embodiment, the model M created as anatomical information in the preoperative plan includes a planned excision surface (planned treatment surface) for excising the tumor TU.

After step S17, the control device 3 executes step S21. In step S21, the control device 3 confirms whether or not the estimated excision surface S estimated in the immediately preceding step S16 has moved significantly compared to the planned excision surface planned in the preoperative plan. When the maximum value of the distance between the two estimated excision surfaces S exceeds a predetermined threshold value, the control device 3 determines that the estimated excision surface S estimated in the immediately preceding step S16 has moved significantly.

When the estimated excision surface S has not moved significantly, the control device 3 performs step S18. When the estimated excision surface S moves significantly, it is possible that the target organ T has moved or the target organ T has been deformed due to excision for some reason. In that case, the control device 3 performs step S11 and step S12.

When the re-execution of the registration step is completed, the control device 3 can convert the coordinate position in the model coordinate system C1 of the model M to the coordinate position in the display coordinate system C2 of the display space according to the actual situation of the target organ T. Next, the control device 3 executes step S13.

According to the surgery assistance system 100B of the present embodiment, the estimated excision surface S can be estimated from the treatment status of the surgeon as in the surgery assistance system 100 according to the first embodiment, and the position information of the tumor TU related to the estimated excision surface S and the vessel information related to the estimated excision surface S can be quickly grasped. In addition, the surgery assistance system 100B performs registration again (reregistration) when the target organ T moves for some reason or the target organ T is deformed due to excision, so that the estimated excision surface S can be estimated in accordance with the situation of the actual target organ T, and the anatomical information related to the estimated excision surface S can be displayed more accurately.

Although the second embodiment of the present invention has been described in detail with reference to the drawings, the specific configuration is not limited to this embodiment and includes design changes and the like within a range not deviating from the gist of the present invention. In addition, the components shown in the above-described embodiments and modifications can be appropriately combined and configured.

### Modification 2

For example, in the above embodiment, the reregistration step modifies the correspondence for converting the coordinate position in the model coordinate system C1 to the coordinate position in the display coordinate system C2, but the reregistration step is not limited to this. The reregistration step may change the data of the model M itself. The reregistration step of changing the model M can cope with a case where the shape of the target organ T itself is greatly deformed, such as a case where the target organ T is greatly opened by an incision.

### Third Embodiment

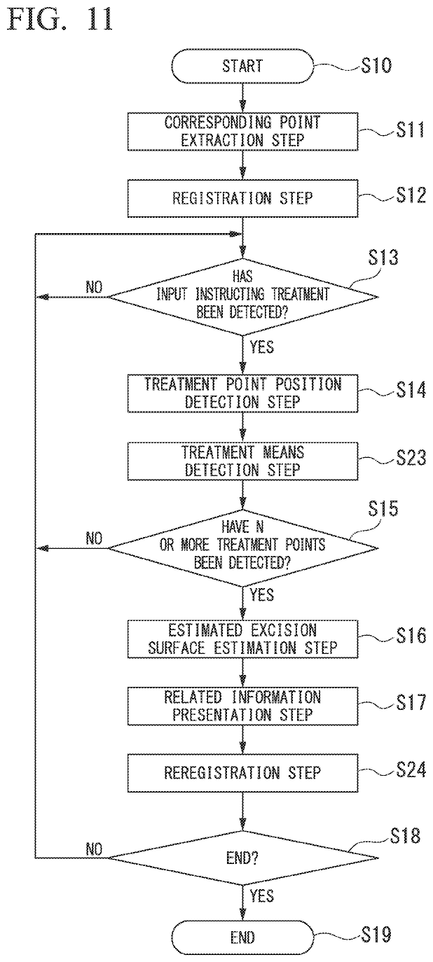

The third embodiment of the present invention will be described with reference to FIGS. 11 to 13. In the following description, the same reference numerals will be given to the configurations common to those already described, and duplicate description will be omitted.

Similar to the surgery assistance system 100 according to the first embodiment, a surgery assistance system 100C according to the present embodiment includes a treatment tool 1, an endoscope 2, a control device 3, a display device 4, an input device 5, and the like. The surgery assistance system 100C differs from the surgery assistance system 100 according to the first embodiment only in the control performed by the control device 3. Hereinafter, a description will be given according to the control flowchart of the control device 3 shown in FIG. 11. The control from step S10 to step S14 is the same as that of the first embodiment.

After step S14, the control device 3 executes step S23. In step S23, the control device 3 detects the means of treatment for the treatment point instructed in step S13 (treatment means detection step). In the present embodiment, the control device 3 detects whether the input instruction is energization by the “incision mode” or the energization by the “hemostatic mode”. The detected treatment means is recorded in the storage portion 36 together with the position of the treatment point P detected in step S14 (treatment means recording step). Next, the control device 3 executes step S15.

After step S17, the control device 3 executes step S24. In step S24, the control device 3 performs registration again to modify the correspondence between the model coordinate system C1 and the display coordinate system C2 (reregistration step). The registration performed in step S24 uses the position of the treatment point P and the treatment means detected in steps S14 and S23.

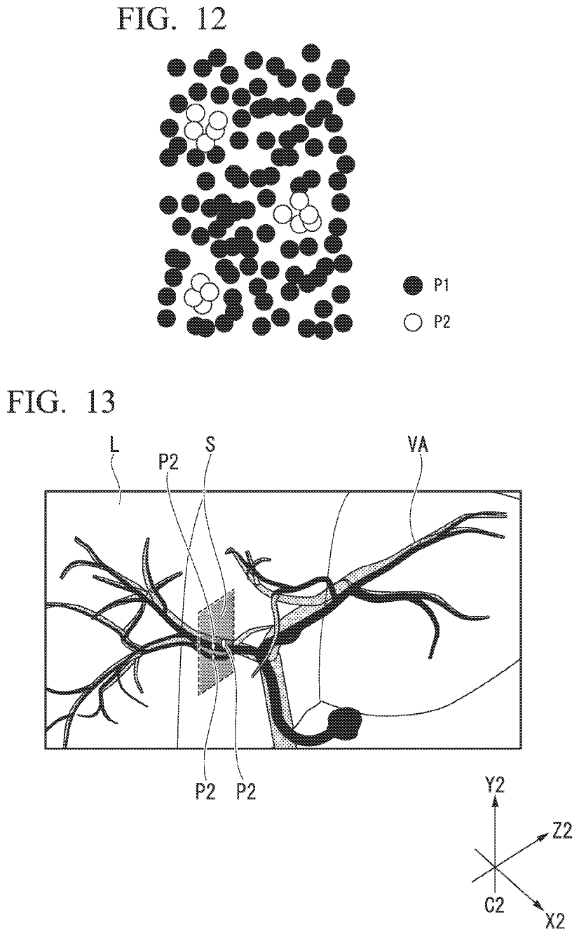

FIG. 12 is a diagram showing the position of the recorded treatment point P and the treatment means, and is a view when viewed perpendicular to the excision surface.

The treatment point P1 that was energized in the “incision mode” is the treatment point where the target organ T was actually incised. On the other hand, the treatment point P2 in which energization was performed in the “hemostatic mode” is a treatment point in which bleeding occurred from the vessel of the target organ T and hemostasis was performed. Therefore, it is highly possible that the vessel of the target organ T is present at the treatment point P2 in which electricity is applied in the “hemostatic mode”.

FIG. 13 is a virtual image VA of a vessel after the reregistration step.

The control device 3 changes the correspondence that converts the coordinate position in the model coordinate system C1 to the coordinate position in the display coordinate system C2, and performs registration so that the coordinate position in the display coordinate system C2 of the vessel matches the treatment point P2. As shown in FIG. 13, the intersection of the virtual image VA of the vessel after the reregistration step and the estimated excision surface S substantially coincides with the treatment point P2.

According to the surgery assistance system 100C of the present embodiment, the estimated excision surface S can be estimated from the treatment status of the surgeon as in the surgery assistance system 100 according to the first embodiment, and the position information of the tumor TU related to the estimated excision surface S and vessel information related to the estimated excision surface S can be quickly grasped. Further, the surgery assistance system 100C estimates the estimated excision surface S according to the actual condition of the target organ T by performing the registration based on the process again, and the anatomical information related to the estimated excision surface S can be displayed more accurately.

Although the third embodiment of the present invention has been described in detail with reference to the drawings, the specific configuration is not limited to this embodiment and includes design changes and the like within a range not deviating from the gist of the present invention. In addition, the components shown in the above-described embodiments and modifications can be appropriately combined and configured.

### Modification 3

For example, in the above embodiment, the treatment tool 1 and the endoscope 2 are manually operated by the surgeon or the scopist, but the mode of the treatment tool or the endoscope is not limited to this. The treatment tool and the endoscope may be operated by a robot arm.

The present invention can be applied to a surgery assistance system that performs treatment using an endoscope.

What is claimed is:

1. A surgery assistance system, comprising:
an endoscope;
a display configured to display an image from the endoscope;
a treatment tool that includes an end effector at a distal end;
an input device configured to input an instruction to the end effector; and
a processor connected to the endoscope, the display, the treatment tool, and the input device,
wherein the processor is configured to
detect a distal end position of the end effector based on the instruction,
record the detected distal end position, and
estimate a first treatment surface from a plurality of recorded distal end positions.

2. The surgery assistance system according to claim 1, wherein
the processor is configured to
make a correspondence between anatomical information of a target organ and the image, and
display on the display the anatomical information related to the first treatment surface that has been estimated.

3. The surgery assistance system according to claim 2, wherein the correspondence is a correspondence between a first coordinate system defined by the anatomical information and a second coordinate system defined by the image.

4. The surgery assistance system according to claim 2, wherein the anatomical information is vessel information near the first treatment surface.

5. The surgery assistance system according to claim 1, wherein
the treatment tool is an energy device, and
the processor is configured to detect the distal end position at a time of the instruction to apply energy to the end effector.

6. The surgery assistance system according to claim 2, wherein
the anatomical information includes a second treatment surface that has been preoperatively planned, and
when a distance between the first treatment surface and the second treatment surface exceeds a predetermined threshold value, the processor modifies the correspondence between the anatomical information and the image.

7. An operating method for a surgery assistance system that includes a treatment tool equipped with an end effector at a distal end, the method comprising:
an anatomical information acquisition step of acquiring anatomical information of a target organ;
a treatment point position detection step of detecting a distal end position of the end effector;
a treatment point position recording step of recording the distal end position that has been detected;
an estimated excision surface estimation step of estimating a first treatment surface from the distal end position that has been recorded; and
a related information presentation step of presenting the anatomical information related to the first treatment surface.

8. The method according to claim 7, further comprising a registration step of making a correspondence between a first coordinate system defined by the anatomical information and a second coordinate system defined by the image from the endoscope.

9. The method according to claim 7, wherein the anatomical information that is presented is vessel information near the first treatment surface.

10. The method according to claim 8, wherein
the anatomical information includes a second treatment surface that has been preoperatively planned, and
when a distance between the second treatment surface and the first treatment surface exceeds a predetermined threshold value, the registration step is performed again.

11. The method according to claim 8, further comprising:
a treatment means detection step of detecting treatment means by the end effector;
a treatment means recording step of recording the detected treatment means together with the distal end position; and
a reregistration step of modifying the correspondence between the first coordinate system and the second coordinate system by using the distal end position and the treatment means.

12. A control device of a surgery assistance system including a treatment tool equipped with an end effector at a distal end, the control device comprising a processor that performs:
an anatomical information acquisition step of acquiring anatomical information of a target organ;
a treatment point position detection step of detecting a distal end position of the end effector;
a treatment point position recording step of recording the distal end position that has been detected;
an estimated excision surface estimation step of estimating a first treatment surface from the distal end position that has been recorded; and
a related information presentation step of presenting the anatomical information related to the first treatment surface.

13. The control device according to claim 12, wherein the processor further performs a registration step of making a correspondence between a first coordinate system defined by the anatomical information and a second coordinate system defined by an image from the endoscope.

14. The control device according to claim 12, wherein the anatomical information that has been presented is vessel information near the first treatment surface.

15. The control device according to claim 13, wherein
the anatomical information includes a second treatment surface that has been preoperatively planned, and
when a distance between the second treatment surface and the first treatment surface exceeds a predetermined threshold value, the registration step is performed again.

16. The control device according to claim 13, wherein the processor further performs:
a treatment means detection step of detecting treatment means by the end effector;
a treatment means recording step of recording the detected treatment means together with the distal end position; and
a reregistration step of modifying the correspondence between the first coordinate system and the second coordinate system by using the distal end position and the treatment means.
